(19) United States
(12) Patent Application Publication
(10) Pub. No.: US 2025/0082518 A1
(43) Pub. Date: Mar. 13, 2025

(54) LIQUID BARRIER AND AIR-PERMEABLE BACKSHEET

(71) Applicant: Berry Global, Inc., Evansville, IN (US)

(72) Inventors: Naizhao Fang, Suzhou (CN); Tianlei Zhang, Suzhou (CN); Yongji Jin, Suzhou (CN)

(21) Appl. No.: 18/830,301

(22) Filed: Sep. 10, 2024

Related U.S. Application Data
(60) Provisional application No. 63/537,779, filed on Sep. 11, 2023.
(60) Provisional application No. 63/667,460, filed on Jul. 3, 2024.

(51) Int. Cl. A61F 13/514; A61F 13/15; A61F 13/51; D04H 1/4291; D04H 1/4382; D04H 1/56; D04H 3/007; D04H 3/16

(52) U.S. Cl. CPC A61F 13/51405; A61F 13/15658; A61F 13/15731; A61F 13/15739; A61F 13/51401; D04H 1/4291; D04H 1/43838; D04H 1/56; D04H 3/007; D04H 3/16; A61F 2013/51023; A61F 2013/51441; A61F 2013/51452; D10B 2321/022; D10B 2401/021; D10B 2509/026

(57) ABSTRACT

Backsheets having a combination of desirable liquid barrier properties and air permeability are provided. The backsheets include outermost spunbond layers and two or more fine-fiber containing nonwoven layers.

### CROSS-REFERENCE TO RELATED APPLICATIONS

This application claims priority under 35 U.S.C. § 119 to U.S. Provisional Application No. 63/537,779 filed Sep. 11, 2023 and U.S. Provisional Application No. 63/667,460 filed Jul. 3, 2024, which is expressly incorporated by reference herein in its entirety.

### TECHNICAL FIELD

Embodiments of the presently-disclosed invention relate generally to backsheets for hygiene articles, such as diapers or the like, that include one or more nonwoven fabrics having a plurality of fine-fiber containing nonwoven layers disposed directly or indirectly between outer spunbond nonwoven layer.

### BACKGROUND

Backsheets of hygienic products, such as diapers, are typically composed of one or more film layers or laminates of a film, such as a polyethylene film, and a nonwoven fabric. The backsheet provides a barrier to liquid, such as urine, blood, body liquid, etc. In some instances, the film of the backsheet may comprise a polyethylene film that may be either microporous or nonporous. These films provides adequate barrier to water, blood, and saline, but they also undesirably prevent air ventilation. Consequently, moisture or wetness gets trapped against the skin of wearer, which is likely to cause a diaper rash, irritated skin, chafing, etc.

In this regard, there remains a need in the art for a backsheet that provides an adequate level of air permeability while simultaneously providing adequate liquid barrier properties.

### SUMMARY OF INVENTION

One or more embodiments of the invention may address one or more of the aforementioned problems. Certain embodiments according to the invention provides a backsheet including at least a first nonwoven fabric. The first nonwoven fabric may comprise the following: (i) a first outermost spunbond layer including a first plurality of continuous spunbond fibers; (ii) a second outermost spunbond layer including a second plurality of continuous spunbond fibers; (iii) a first fine-fiber containing layer including a first plurality of fine fibers, in which the first fine-fiber containing layer is located proximately or adjacent to the first outermost spunbond layer; and (iv) a second fine-fiber containing layer including a second plurality of fine fibers, in which the second fine-fiber containing layer may be located proximately or adjacent to the second outermost spunbond layer and adjacent to the first fine-fiber containing layer. In accordance with certain embodiments of the invention, a total amount of fine fibers comprising the first plurality of fine fibers and the second plurality of fine fibers account for about 15 to about 40% by weight of the first nonwoven fabric, such as at least about any of the following: 15, 16, 18, 20, 22, 24, 25, 26, 28, and 30% by weight of the first nonwoven fabric, and/or at most about any of the following: 40, 38, 36, 35, 34, 32, and 30% by weight of the first nonwoven fabric.

In another aspect, the present invention provides a hygiene article including (i) a topsheet, (ii) an absorbent core, and (iii) a backsheet, such as any of those disclosed and described herein, in which the absorbent core is located directly or indirectly between the topsheet and the backsheet.

In another aspect, the present invention provides A method of making a backsheet comprising forming at least a first nonwoven fabric, in which forming the first nonwoven fabric may comprise the following steps: (i) providing or forming a first outermost spunbond layer including a first plurality of continuous spunbond fibers; (ii) providing or forming a second outermost spunbond layer including a second plurality of continuous spunbond fibers; (iii) providing or forming a first fine-fiber containing layer including a first plurality of fine fibers; (iv) providing or forming a second fine-fiber containing layer including a second plurality of fine fibers; (v) positioning the first fine-fiber containing layer proximately or adjacent to the first outermost spunbond layer, and positioning the second fine-fiber containing layer is proximately or adjacent to the second outermost spunbond layer and adjacent to the first fine-fiber containing layer to form a first intermediate material; and (vi) consolidating the first intermediate material to form a backsheet, such as those disclosed and described herein.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a backsheet including a single nonwoven fabric in accordance with certain embodiments of the invention;

FIG. 2 illustrates a backsheet including two woven fabrics thermally bonded to each other in accordance with certain embodiments of the invention;

FIG. 3 illustrates one particular backsheet according to the general depiction of FIG. 2;

FIG. 4 illustrates a backsheet including two woven fabrics a bonded adhesively to each other in accordance with certain embodiments of the invention;

FIG. 5 illustrates one particular backsheet according to the general depiction of FIG. 4;

FIG. 6 illustrates a backsheet having a single nonwoven fabric and a water-repellant coating applied to both sides thereof in accordance with certain embodiments of the invention;

FIG. 7 illustrates a backsheet having two nonwoven fabrics bonded together and a water-repellant coating applied to both sides thereof in accordance with certain embodiments of the invention;

FIG. 8 illustrates a cross-sectional view of a hygiene article in accordance with certain embodiments of the invention.

### DETAILED DESCRIPTION

The invention now will be described more fully hereinafter. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to backsheets for hygiene articles, such as diapers or the like, that include one or more nonwoven fabrics having a plurality of fine-fiber containing nonwoven layers disposed directly or indirectly between outer spunbond nonwoven layer. In accordance with certain embodiments of the invention, by way of example, the backsheet may comprises one or more nonwoven fabrics having a spunbond-meltblown-spunbond (SMS) structure, such as SₓM_{y}S_{z} where ‘x’ represents the number of spunbond layers and may be 1, 2, 3, 4, or 5; ‘y’ represents the number of meltblown layers and may be from 2, 3, 4, 5, 6, 7, and 8; and ‘z’ represents the number of spunbond layers and may be 1, 2, 3, 4, or 5. In accordance with certain embodiments, the fine-fibers may include meltblown fibers, melt-fibrillated fibers and/or electrospun fibers as noted later. The backsheets in accordance with certain embodiments of the invention may simultaneously provide adequate or improved liquid barrier properties (e.g., large hydrostatic head values) and adequate or improved air permeability values (e.g., large CFM values). In this regard, hygiene articles including a backsheet as described and disclosed herein may prevent bodily fluids from a wearer from passing or leaking through the article while also providing sufficient vapor breathability to mitigate or prevent the wearer from experiencing diaper rash, irritated skin, chafing, etc.

In accordance with certain embodiments of the invention, the backsheet may be formed from fibers (e.g., at least a portion thereof) formed from one or more polymer melts including a masterbatch additive including a water-repellent additive (e.g., additive compound(s) rendering a surface hydrophobic so as to repel water and other polar compounds or lowering of the surface tension at the surface) and/or the backsheet may be surface treated with a water-repellent composition (e.g., composition rendering a surface hydrophobic so as to repel water and other polar compounds or lowering of the surface tension at the surface) or on one or both outermost surfaces thereof. In accordance with certain embodiments of the invention, one or more (e.g., all) of the individual nonwoven layers of the backsheet may be formed from respective polymer melts including a masterbatch additive including a water-repellent additive (e.g., additive compound(s) rendering a surface hydrophobic so as to repel water and other polar compounds or lowering of the surface tension at the surface. By way of example only, the backsheet may comprises one or more SMS-type nonwoven fabrics in which one or both outermost surfaces have been subjected to a surface treatment is to reduce surface tension and enhance the barrier to liquid penetration. The treatment chemicals, whether provided as part of a masterbatch or as a surface treatment, are not particularly limited but can include fluoride, silicones etc. The backsheet, for example, may have an air permeability of 18 CFM or greater (test method: Area: 38 cm², 125 Pa); a small pore size (e,g., 5˜12 microns tested by Proximeter); resistant to saline penetration; resistant to water penetration; resistant to alcohol penetration (e.g., Alcohol repellent ≥5 rating); resistant to blood penetration; and have a hydrostatic head value of 65 mbar or greater (test method: Saline water, 500 mbar/min).

Alternatively, the backsheet may be devoid of any such surface treatment with a water-repellent composition. The backsheet, for example, may have an air permeability of 18 CFM or greater (test method: Area: 38 cm², 125 Pa); a small pore size (e,g, 5˜12 microns tested by Proximeter); resistant to saline penetration; resistant to water penetration; resistant to alcohol penetration (e.g., Alcohol repellent ≥5 rating); resistant to blood penetration; and have a hydrostatic head value of 65 mbar or greater (test method: Saline water, 500 mbar/min).

The terms “nonwoven” and “nonwoven web”, as used herein, may comprise a web having a structure of individual fibers, fibers, and/or threads that are interlaid but not in an identifiable repeating manner as in a knitted or woven fabric. Nonwoven webs, according to certain embodiments of the invention, may be formed by any process conventionally known in the art such as, for example, meltblowing processes, spunbonding processes, air-laid, and carded web processes. A “nonwoven web”, as used herein, may comprise a plurality of individual fibers that have not been subjected to a consolidating process. In certain instances, the “nonwoven web” may comprises a plurality of layers, such as one or more spunbond layers and/or one or more meltblown layers. For instance, a “nonwoven web” may comprises a spunbond-meltblown-spunbond structure.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples. Furthermore, the term “consolidated” and “consolidation” may comprise the bonding by means of a through-air-bonding operation. The term “through-air bonded” and “though-air-bonding”, as used herein, may comprise a nonwoven web consolidated by a bonding process in which hot air is used to fuse the fibers at the surface of the web and optionally internally within the web. By way of example only, hot air can either be blown through the web in a conveyorized oven or sucked through the web as it passes over a porous drum as a vacuum is developed. The temperature of and the rate of hot air are parameters that may determine the level or the extent of bonding in nonwoven web. In accordance with certain embodiments of the invention, the temperature of the hot air may be high enough to melt, induce flowing, and/or fuse the a plurality of fibers having a lower melting point temperature or onset of lower melting point temperature (e.g., amorphous fibers) to a plurality of fibers having a higher melting point temperature or onset of lower melting point temperature (e.g., semi-crystalline or crystalline fibers). Such a web may be considered a “consolidated nonwoven”, “nonwoven fabric” or simply as a “fabric” according to certain embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The term “elastic”, “elastomer”, “elastomeric polymer” or “elastomeric”, as used interchangeably herein, may comprise a material (e.g., a nonwoven layer or layers) that when stretched and released will recover to near its original length (e.g., return to within 20%, 10%, 5%, 3%, or 1% of its original length). The term “elastic”, “elastomer”, or “elastomeric”, as used interchangeably herein, may also comprise a material that exhibits the ability to be stretched and released several times and, to exert repetitively the same or just slightly lower force when stretched at the same extension level. Elastic materials, for example, may comprise elastomers, such as elastomeric polymers. Non-limiting exemplary elastomers may comprise, according to certain embodiments, one or more elastomers such as an acrylate; a polyolefin, such as polyethylene, polypropylene, polybutylene, polyhexene, polyoctene; polystyrenes; polyurethanes; polyesters, such as polyethyleneterephthalate; polyamides such as nylon; natural or synthetic rubber resins such as styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof. Additionally or alternatively, non-limiting elastomers may include elastomeric polyolefins (e.g., VISTAMAXX™ from ExxonMobil Chemical Company, VERSIFY™, a propylene-ethylene elastomeric polymer, and AFFINITY™ from The Dow Chemical Company), polyether block amide copolymer (e.g., PEBAX® from Arkema Group), polyester block amide copolymer, copolyester thermoplastic elastomer (e.g., ARNITEL® from DSM Engineering Plastics, HYTREL® from E.I. DuPont de Nemours and Company), thermoplastic urethane elastomer, and/or combinations thereof. In certain embodiments, example elastomers may comprise VISTAMAXX™ propylene-based elastomers (commercially available form ExxonMobile), which comprise copolymers of propylene and ethylene. VISTAMAXX™ propylene-based elastomers, for example, comprise isotactic polypropylene microcrystalline regions and random amorphous regions.

“Elastic stretch,” “elasticity,” or variants thereof, means the degree to which a film or nonwoven fabric may be stretched before breaking or becoming permanently deformed. Elastic stretch is typically expressed as a percentage of the original length. For example, an elastic stretch of 100% means that a film or nonwoven fabric may be stretched to about twice its original length before breaking or permanent deformation.

The term “non-elastomeric polymers” may refer to polymers that when stretched and released do not recover to near its original length (e.g., return to within 20%, 10%, 5%, 3%, or 1% of its original length) and/or have an elastic stretch below 100%, such as below 80%, 50%, or 20%.

The term “spunbond”, as used herein, may comprise fibers which are formed by extruding molten thermoplastic material as filaments from a plurality of fine, usually circular, capillaries of a spinneret with the diameter of the extruded filaments then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®. Spunbond fibers, for example, comprise continuous fibers.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

The term “melt fibrillation”, as used herein, may comprise a general class of making fibers defined in that one or more polymers are molten and may be extruded into many possible configurations (e.g. co-extrusion, homogeneous or bicomponent films or filaments) and then fibrillated or fiberized into a plurality of individual filaments for the formation of melt-fibrillated fibers. Non limiting examples of melt-fibrillation methods may include melt blowing, melt fiber bursting, and melt film fibrillation. The term “melt-film fibrillation”, as used herein, may comprise a method in which a melt film is produced from a melt and then a fluid is used to form fibers (e.g., melt-film fibrillated fibers) from the melt film. Examples include U.S. Pat. Nos. 6,315,806, 5,183,670, 4,536,361, 6,382,526, 6,520,425, and 6,695,992, in which the contents of each are incorporated by reference herein to the extent that such disclosures are consistent with the present disclosure. Additional examples include U.S. Pat. Nos. 7,628,941, 7,722,347, 7,666,343, 7,931,457, 8,512,626, and 8,962,501, which describe the Arium™ melt-film fibrillation process for producing melt-film fibrillated fibers (e.g., having sub-micron fibers).

The term “machine direction” or “MD”, as used herein, comprises the direction in which the fabric produced or conveyed. The term “cross-direction” or “CD”, as used herein, comprises the direction of the fabric substantially perpendicular to the MD.

The term “layer”, as used herein, may comprise a generally recognizable combination of similar material types and/or functions existing in the X-Y plane.

Certain embodiments according to the invention provides a backsheet including at least a first nonwoven fabric. The first nonwoven fabric may comprise the following: (i) a first outermost spunbond layer including a first plurality of continuous spunbond fibers; (ii) a second outermost spunbond layer including a second plurality of continuous spunbond fibers; (iii) a first fine-fiber containing layer including a first plurality of fine fibers, in which the first fine-fiber containing layer is located proximately or adjacent to the first outermost spunbond layer; and (iv) a second fine-fiber containing layer including a second plurality of fine fibers, in which the second fine-fiber containing layer may be located proximately or adjacent to the second outermost spunbond layer and adjacent to the first fine-fiber containing layer. In accordance with certain embodiments of the invention, a total amount of fine fibers comprising the first plurality of fine fibers and the second plurality of fine fibers account for about 15 to about 40% by weight of the first nonwoven fabric, such as at least about any of the following: 15, 16, 18, 20, 22, 24, 25, 26, 28, and 30% by weight of the first nonwoven fabric, and/or at most about any of the following: 40, 38, 36, 35, 34, 32, and 30% by weight of the first nonwoven fabric.

In accordance with certain embodiments of the invention, a total amount of continuous spunbond fibers comprising the first plurality of continuous spunbond fibers and the second plurality of continuous spunbond fibers (plus fine fibers from any other additional fine-fiber containing layers located between the outer spunbond layers) account for about 60 to about 85% by weight of the first nonwoven fabric, such as at least about any of the following: 60, 62, 64.65. 66, 68, and 70% by weight of the first nonwoven fabric, and/or at most about any of the following: 85, 84, 82, 80, 78, 76, 74, 72, and 70% by weight of the first nonwoven fabric. Additionally or alternatively, the first plurality of continuous spunbond fibers, the second plurality of continuous spunbond fibers, or both independently from each other have an average diameter from about 10 to about 25 microns, such as at least about any of the following: 10, 13, 15, and 18 microns, and/or at most about any of the following: 25, 22, 20, and 18 microns. In this regard, the first plurality of continuous spunbond fibers may have a different average diameter than the second plurality of continuous spunbond fibers given that one of the spunbond layers may be proximate or adjacent an absorbent core while the other spunbond layer may be proximate or adjacent an external environment.

The first outermost spunbond layer, the second outermost spunbond layer, or both may independently from each other comprises a spunbond polymeric composition including (i) a spunbond-polymer component, and optionally (ii) a spunbond-additive component. For instance, the spunbond-polymer component may comprise one or more non-elastomeric polymers. By way of example, the one or more non-elastomeric polymers may comprise a non-elastic polyolefin, a non-elastic polyester, a non-elastic polyamide, or blends thereof. In accordance with certain embodiments of the invention, the non-elastic polyolefin may comprise a polypropylene, a propylene-containing copolymer, polyethylene, an ethylene-containing copolymer, and any combination thereof. Additionally or alternatively, the spunbond-polymer component comprises from 60 to 100% by weight of the one or more non-elastomeric polymers, which may optionally comprise a polypropylene, a propylene-containing copolymer, polyethylene, an ethylene-containing copolymer, and any combination thereof.

In accordance with certain embodiments of the invention, the spunbond-additive component may comprise one or more melt-additives. For instance, the spunbond-additive component may comprise a softness enhancer comprising an elastomeric polymer or polymers. In this regard, the softness enhancer may be distinguished from the spunbond-polymer component, at least, on the basis that the softness enhancer is an elastomeric polymer or polymers, while the spunbond-polymer component may be non-elastomeric in nature. In accordance with certain embodiments of the invention, the elastomeric polymer comprises an elastomeric polyolefin, such as a propylene-ethylene copolymer, a polyether block amide copolymer, a polyester block amide copolymer, a copolyester thermoplastic elastomer, or any combination thereof.

Non-limiting example elastomeric polymers may comprise, according to certain embodiments, styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof. Additionally or alternatively, non-limiting elastomeric polymers may include elastomeric polyolefins (e.g., VISTAMAXX™ from ExxonMobil Chemical Company, VERSIFY™, a propylene-ethylene elastomeric polymer, and AFFINITY™ from The Dow Chemical Company), polyether block amide copolymer (e.g., PEBAX® from Arkema Group), polyester block amide copolymer, copolyester thermoplastic elastomer (e.g., ARNITEL® from DSM Engineering Plastics, HYTREL® from E.I. DuPont de Nemours and Company), thermoplastic urethane elastomer, and/or combinations thereof. In certain embodiments, example elastomeric polymers may comprise VISTAMAXX™ propylene-based elastomers (commercially available form ExxonMobile), which comprise copolymers of propylene and ethylene. VISTAMAXX™ propylene-based elastomers, for example, comprise isotactic polypropylene microcrystalline regions and random amorphous regions.

In accordance with certain embodiments of the invention, the first plurality of continuous spunbond fibers, the second plurality of continuous spunbond fibers, or both independently from each other have from about 2 to about 20% by weight of the elastomeric polymer, such as at least about any of the following: 2, 3, 4, 5, 6, 7, 8, 9, and 10% by weight, and/or at most about any of the following: 20, 19, 18, 17, 16, 15, 14, 13, 12, 11, and 10% by weight. Additionally or alternatively, the first nonwoven fabric has from about 0.01 to about 5% by weight of the elastomeric polymer, such as at least about any of the following: 0.01, 0.03, 0.05, 0.08, and 0.1% by weight, and/or at most about any of the following: 5, 3, 2, 1. 0.5, 0.2, 0.18, 0.15, 0.12, and 1% by weight.

In accordance with certain embodiments of the invention, the spunbond-additive component may comprise a slip additive comprising one or more amides, such as one or more primary amides, such as erucamide, oleamide, strearamide, behenamide, one or more bis-amides, such as ethylene bis-amide, or any combination thereof.

In accordance with certain embodiments of the invention, the slip additive comprises one or more amides, in which the one or more amides comprises an unsaturated aliphatic chain, a saturated aliphatic chain, or a combination thereof. In accordance with certain embodiments of the invention, the one or more aliphatic chains may each independently comprise from about 1 to about 30 carbon atoms (e.g., about 5 to about 30 carbon atoms). For example, a secondary amides and bis-amides may comprise two saturated and/or unsaturated carbon chains the may each independently comprise from about 1 to about 30 carbon atoms (e.g., about 5 to about 30 carbon atoms). By way of example only, the one or more aliphatic chains may each independently comprise from at least about any of the following: 1, 5, 10, 15, 16, 17, 18, 19, 20, 21, 22, 23, 24, and 25 carbon atoms and/or at most about 30, 29, 28, 27, 26, 25, 20, and 15 carbon atoms (e.g., about 15 to about 25 carbon atoms, about 20 to 30 carbon atoms, etc.). In accordance with certain embodiments of the invention, the slip additive may comprise an amide including an unsaturated aliphatic chain having one or more elements or unsaturation. An element of unsaturation corresponds to two fewer hydrogen atoms than in the saturated formula. For example, a single double bound accounts for one element of unsaturation, while a triple bond would account for two elements of unsaturation. In accordance with certain embodiments of the invention, the slip additive includes an unsaturated aliphatic chain comprising from about 1 to about 10 elements of unsaturation (e.g., 1, 2, 3, 4, 5, 6, 7, 8, 9, or 10 elements of saturation).

In accordance with certain embodiments of the invention, the slip additive may comprise a combination of a greater amount of, for example, stearamide and a lesser amount of, for example, erucamide. For example, the combination of the greater amount of stearamide and the lesser amount of erucamide may comprise from about 25 to about 40 weight percent erucamide and from about 60 to about 75 weight percent stearamide.

The first plurality of continuous spunbond fibers, the second plurality of continuous spunbond fibers, or both independently from each other may have from 0.1 to 5% by weight of the amide(s), such as at least about any of the following: 0.1, 0.2, 0.4, 0.5, 0.6, 0.8, 1, 1.2, 1.4, 1.5, 1.6, 1.8, and 2 wt. %, and/or at most about any of the following: 5, 4.8, 4.5, 4.2, 4, 3.8, 3.5, 3.2, 3, 2.8, 2.5, 2.2, and 2 wt. %. Additionally or alternatively, the first nonwoven fabric has from about 0.005 to about 0.5% by weight of the amide(s), such as at least about any of the following: 0.005, 0.008, 0.01, 0.15, and 0.02% by weight, and/or at most about any of the following: 5, 3, 1, 0.5, 0.1, 0.05, 0.045, 0.040, 0.035, 0.030, 0.025, and 0.02% by weight.

In accordance with certain embodiments of the invention, the first plurality of continuous spunbond fibers, the second plurality of continuous spunbond fibers, or both independently from each other have a round cross-section or a non-round cross-section. For example, at least the first plurality of continuous spunbond fibers, the second plurality of continuous spunbond fibers, or both independently from each other may have from 10 to 100% by number of non-round cross-sectional fibers, such as at least about any of the following: 10, 20, 30, 40, and 50% by number, and/or at most about any of the following: 100, 95, 90, 85, 80, 70, 60, and 50% by number. Additionally or alternatively, at least the first plurality of continuous spunbond fibers, the second plurality of continuous spunbond fibers, or both independently from each other may have from 10 to 100% by number of round cross-sectional fibers, such as at least about any of the following: 10, 20, 30, 40, and 50% by number, and/or at most about any of the following: 100, 95, 90, 85, 80, 70, 60, and 50% by number. In accordance with certain embodiments of the invention, round cross-sectional fibers may have an aspect ratio from 0.8:1 to 1.2:1, such as at least about any of the following: 0.8:1, 0.9:1, and 1:1, and/or at most about any of the following: 1.2:1, 1.1:1, and 1:1. In accordance with certain embodiments of the invention, non-round cross-sectional fibers may have an aspect ratio from about 1.5:1 to about 10:1, such as at least about any of the following: 1.5:1, 2:1, 3:1, 4:1, and 5:1, and//or at most about any of the following: 10:1, 9:1, 8:1, 7:1, 6:1, and 5:1. As used herein, the term “aspect ratio” comprises a ratio of the length of the major axis to the length of the minor axis of the cross-section of the fiber in question.

In accordance with certain embodiments of the invention, the first plurality of fine fibers, the second plurality of fine fibers, or both independently from each other may have an average diameter from about 0.2 to about 25 microns, such as at least about any of the following: 0.2, 0.5, 0.8, 1, 1.5, 1.8, and 2 microns, and/or at most about any of the following: 5, 4.5, 4, 3.5, 3, 2.5 and 2 microns. Additionally or alternatively, the first plurality of fine fibers, the second plurality of fine fibers, or both independently from each other comprise meltblown fiber, melt-fibrillated fibers, and/or electrospun fibers.

The first fine-fiber containing layer, the second fine-fiber containing layer, or both independently from each other comprises a fine-fiber polymeric composition including (i) a fine-fiber-polymer component, and optionally (ii) a fine-fiber-additive component. For instance, the fine-fiber-polymer component may comprise one or more non-elastomeric polymers. By way of example, the one or more non-elastomeric polymers may comprise a non-elastic polyolefin, a non-elastic polyester, a non-elastic polyamide, or blends thereof. In accordance with certain embodiments of the invention, the non-elastic polyolefin may comprise a polypropylene, a propylene-containing copolymer, polyethylene, an ethylene-containing copolymer, and any combination thereof. Additionally or alternatively, the fine-fiber-polymer component comprises from 60 to 100% by weight of the one or more non-elastomeric polymers, which may optionally comprise a polypropylene, a propylene-containing copolymer, polyethylene, an ethylene-containing copolymer, and any combination thereof.

In accordance with certain embodiments of the invention, the s fine-fiber-additive component may comprise one or more melt-additives. For instance, the fine-fiber-additive component may comprise a softness enhancer comprising an elastomeric polymer or polymers. In this regard, the softness enhancer may be distinguished from the fine-fiber-polymer component, at least, on the basis that the softness enhancer is an elastomeric polymer or polymers, while the fine-fiber-polymer component may be non-elastomeric in nature. In accordance with certain embodiments of the invention, the elastomeric polymer comprises an elastomeric polyolefin, such as a propylene-ethylene copolymer, a polyether block amide copolymer, a polyester block amide copolymer, a copolyester thermoplastic elastomer, or any combination thereof.

Non-limiting example elastomeric polymers, as noted above, may comprise, according to certain embodiments, styrenic block copolymers (e.g., styrene-isoprene-styrene, styrene-butadiene-styrene, styrene-ethylene-ethylene-propylene-styrene copolymers, styrene-ethylene-butylene-styrene); epoxies; vinyl acetates, such as ethylene vinyl acetate; polydiorganosiloxane polyurea copolymers; copolymers thereof and mixtures thereof. Additionally or alternatively, non-limiting elastomeric polymers may include elastomeric polyolefins (e.g., VISTAMAXX™ from ExxonMobil Chemical Company, VERSIFY™, a propylene-ethylene elastomeric polymer, and AFFINITY™ from The Dow Chemical Company), polyether block amide copolymer (e.g., PEBAX® from Arkema Group), polyester block amide copolymer, copolyester thermoplastic elastomer (e.g., ARNITEL® from DSM Engineering Plastics, HYTREL® from E.I. DuPont de Nemours and Company), thermoplastic urethane elastomer, and/or combinations thereof. In certain embodiments, example elastomeric polymers may comprise VISTAMAXX™ propylene-based elastomers (commercially available form ExxonMobile), which comprise copolymers of propylene and ethylene. VISTAMAXX™ propylene-based elastomers, for example, comprise isotactic polypropylene microcrystalline regions and random amorphous regions.

In accordance with certain embodiments of the invention, the first plurality of fine fibers, the second plurality of fine fibers, or both independently from each other may have from about 0 to about 20% by weight of the elastomeric polymer, such as at least about any of the following: 0, 1, 2, 3, 4, 5, 6, 7, 8, 9, and 10% by weight, and/or at most about any of the following: 20, 19, 18, 17, 16, 15, 14, 13, 12, 11, and 10% by weight. In accordance with certain embodiments of the invention, the fine-fibers may be devoid of an elastomeric polymer.

In accordance with certain embodiments of the invention, the fine-fiber-additive component may comprise a slip additive comprising one or more amides, such as one or more primary amides, such as erucamide, oleamide, strearamide, behenamide, one or more bis-amides, such as ethylene bis-amide, or any combination thereof.

In accordance with certain embodiments of the invention, the slip additive comprises one or more amides, in which the one or more amides comprises an unsaturated aliphatic chain, a saturated aliphatic chain, or a combination thereof. In accordance with certain embodiments of the invention, the one or more aliphatic chains may each independently comprise from about 1 to about 30 carbon atoms (e.g., about 5 to about 30 carbon atoms). For example, a secondary amides and bis-amides may comprise two saturated and/or unsaturated carbon chains the may each independently comprise from about 1 to about 30 carbon atoms (e.g., about 5 to about 30 carbon atoms). By way of example only, the one or more aliphatic chains may each independently comprise from at least about any of the following: 1, 5, 10, 15, 16, 17, 18, 19, 20, 21, 22, 23, 24, and 25 carbon atoms and/or at most about 30, 29, 28, 27, 26, 25, 20, and 15 carbon atoms (e.g., about 15 to about 25 carbon atoms, about 20 to 30 carbon atoms, etc.). In accordance with certain embodiments of the invention, the slip additive may comprise an amide including an unsaturated aliphatic chain having one or more elements or unsaturation. An element of unsaturation corresponds to two fewer hydrogen atoms than in the saturated formula. For example, a single double bound accounts for one element of unsaturation, while a triple bond would account for two elements of unsaturation. In accordance with certain embodiments of the invention, the slip additive includes an unsaturated aliphatic chain comprising from about 1 to about 10 elements of unsaturation (e.g., 1, 2, 3, 4, 5, 6, 7, 8, 9, or 10 elements of saturation).

In accordance with certain embodiments of the invention, the slip additive may comprise a combination of a greater amount of, for example, stearamide and a lesser amount of, for example, erucamide. For example, the combination of the greater amount of stearamide and the lesser amount of erucamide may comprise from about 25 to about 40 weight percent erucamide and from about 60 to about 75 weight percent stearamide.

In accordance with certain embodiments of the invention, the first plurality of fine fibers, the second plurality of fine fibers, or both independently from each other may have from 0 to 5% by weight of the amide(s), such as at least about any of the following: 0, 0.1, 0.2, 0.4, 0.5, 0.6, 0.8, 1, 1.2, 1.4, 1.5, 1.6, 1.8, and 2 wt. %, and/or at most about any of the following: 5, 4.8, 4.5, 4.2, 4, 3.8, 3.5, 3.2, 3, 2.8, 2.5, 2.2, and 2 wt. %. In accordance with certain embodiments of the invention, the fine fibers may be devoid of a slip additive (e.g., one or more additives).

In accordance with certain embodiments of the invention, the first nonwoven fabric may have a basis weight from about 10 to about 100 gsm, such as at least about any of the following: 10, 15, 20, 25, 30, 34, 40, 45, and 50 gsm, and/or at most about any of the following: 100, 80, 60, and 50 gsm.

The backsheet, in accordance with certain embodiments of invention, may comprise more than the first nonwoven fabric, such as 2, 3, 4, or 5 nonwoven fabrics bonded together, in which each of the nonwoven fabrics may comprise a structure and composition as disclosed for the first nonwoven fabric. In accordance with certain embodiments of the invention, the backsheet further comprises a second nonwoven fabric bonded to the first nonwoven fabric, such as by one or more thermal bond sites or via a layer of adhesive located including an adhesive composition between the first nonwoven fabric and the second nonwoven fabric. In accordance with certain embodiments of the invention, the second nonwoven fabric may comprise any nonwoven fabric as disclosed for the first nonwoven fabric.

In accordance with certain embodiments of the invention, the adhesive layer (if present) may have a discontinuous pattern having one or more regions devoid of the adhesive composition. For example, the discontinuous pattern may include a plurality of discrete zones of the adhesive composition surrounded by a continuous zone devoid of the adhesive composition. Alternatively, for example, the discontinuous pattern may include a plurality of discrete zones of the adhesive composition and a plurality of discrete zones devoid of the adhesive composition provided in alternating fashion. Additionally or alternatively, the plurality of discrete zones of the adhesive composition define an adhesive area from about 3 to about 80% of a first surface of the first nonwoven fabric, such as at least about any of the following: 3, 5, 8, 10, 12, 15, 18, 20, 25, 30, 35, and 40%, and/or at most about any of the following: 80, 75, 70, 65, 60, 55, 50, 45, and 40%. Additionally or alternatively, the layer of adhesive may have a basis weight from about 0.1 to about 8 gsm, such as at least about any of the following: 0.1, 0.3, 0.5, 0.8, 1, 1.5, 2, 2.5, 3, 3.5, and 4 gsm, and/or at most about any of the following: 8, 7, 6, 5, and 4 gsm.

In accordance with certain embodiments of the invention, the backsheet comprises one of the following structures:
(Structure 1) S1ₐ-M_{b}-S2_{c};
(Structure 2) S1ₐ-N_{d}-S2_{c};
(Structure 3) S1ₐ-M_{b}-N_{d}-S2_{c};
(Structure 4) S1ₐ-N_{d}-M_{b}-N_{d}-S2_{c};
(Structure 5) S1ₐ-M_{b}-N_{d}-M_{b}-S2_{c}; or any combinations thereof;
wherein
‘M’ comprises a meltblown layer or a melt-fibrillated layer;
‘N’ comprises a sub-micron fiber-containing layer, such as electrospun fibers;
‘S1’ comprises a first spunbond layer;
‘S2’ comprises a second spunbond layer;
‘a’ represents the number of layers and is independently selected from 1, 2, 3, 4, and 5;
‘b’ represents the number of layers is independently selected from 1, 2, 3, 4, 5, 6, 7, and 8;
‘c’ represents the number of layers is independently selected from 1, 2, 3, 4, and 5; and
‘d’ represents the number of layers is independently selected from 1, 2, 3, 4, 5, 6, 7, and 8;
‘e’ represents the number of layers is independently selected from 1, 2, 3, 4, and 5.

By way of example only, the backsheet may comprise the first nonwoven fabric that has a structure according to Structure 1, and wherein ‘a’ is 1 or 2, ‘b’ is 3, 4, or 5, and ‘c’ is 1 or 2. In another embodiment, the backsheet includes the first nonwoven above in combination with a second nonwoven fabric that has a structure according to Structure 1, and wherein ‘a’ is 1 or 2, ‘b’ is 3, 4, or 5, and ‘c’ is 1 or 2.

In accordance with certain embodiments of the invention, the backsheet may have a basis weight from about 10 to about 200 gsm, such as at least about any of the following: 10, 15, 20, 25, 30, 34, 40, 45, 50, 60, 80, and 100 gsm, and/or at most about any of the following: 200, 180, 150, 120, and 100 gsm.

In accordance with certain embodiments of the invention, the backsheet may further comprise a water-repellent surface treatment and/or inclusion of a masterbatch additive to one or more polymer melts forming at least some (or all) of the fibers forming the backsheet, in which the masterbatch additive includes a water-repellent additive (e.g., additive compound(s) rendering a surface hydrophobic so as to repel water and other polar compounds or lowering of the surface tension at the surface), as noted previously. For example, a water-repellent surface treatment may be deposited onto a first outermost surface, a second outermost surface, or both. The water-repellent composition is not particularly limited and may include, for example, a silicone-based composition and/or a fluorine-containing composition including a fluorochemical. In accordance with certain embodiments of the invention, the fibers (e.g., a portion thereof or all) may be formed from polymer melt(s) including a masterbatch and the surface(s) may be topically treated with a water repellant composition.

In accordance with certain embodiments of the invention, the backsheet is devoid of a film.

In accordance with certain embodiments of the invention, the backsheet has an air permeability from about 15 to 60 CFM according to IST70.1, such as at least about any of the following: 15, 18, 20, 23, 25, 28, and 30 CFM according to IST70.1, and/or at most about any of the following: 60, 58, 55, 52, 50, 48, 45, 42, 40, 38, 35, 32, and 30 CFM according to IST70.1. Additionally or alternatively, the backsheet has a first ratio between the air permeability in CFM according to IST70.1 to basis weight in gsm from about 0.3:1 to about 2.5:1, such as at least about any of the following: 0.3:1, 0.4:1, 0.6:1, 0.8:1, and 1:1, and/or at most about any of the following: 2.5:1, 2.3:1, 2:1, 1.8:1, 1.5:1, 1.2:1, and 1:1.

In accordance with certain embodiments of the invention, the backsheet has a hydrostatic head of at least 70 mbar according to Impact Test #1 (described below), such as at least about any of the following: 70, 72, 75, 78, 80, 82, 85, 88, 90, 92, 95, 98, and 100 mbar, and/or at most about any of the following: 125, 122, 120, 118, 115, 112, 110, 108, 105, 102, and 100 mbar. Additionally or alternatively, the backsheet has a second ratio between the hydrostatic head according to Impact Test #1 to basis weight in gsm from about 1.2:1 to about 2.5:1, such as at least about any of the following: 1.2:1, 1.5:1, and 1.8:1, and/or at most about any of the following: 2.5:1, 2.2:1, 2:1, and 1.8:1.

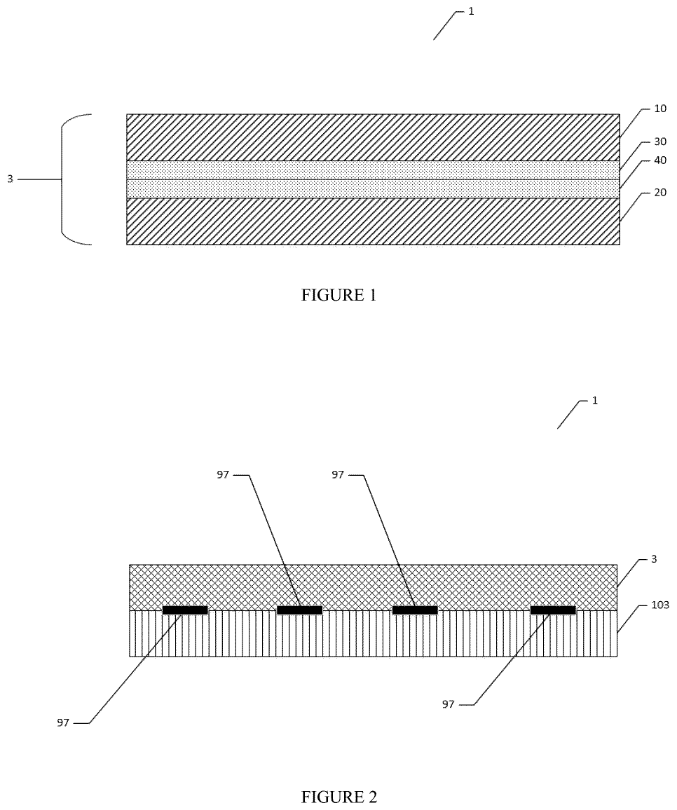

FIG. 1 illustrates a backsheet 1 including a first nonwoven fabric 3 in accordance with certain embodiments of the invention. The first nonwoven fabric 3 includes a first outermost spunbond layer 10 and a second outermost spunbond layer 20, with a first fine-fiber containing layer 30 and a second fine-fiber containing layer 40 located between the spunbond layers. FIG. 2 illustrates a backsheet 1 including a first nonwoven fabric 3 and a second nonwoven fabric 103 thermally bonded to each other via thermal bond sites 97. Although the thermal bond sites 97 are illustrated as shallow bond sites, the thermal bond sites may extend through the entire thickness of the backsheet or completely through one of the nonwoven fabrics and only partially through the other nonwoven fabric.

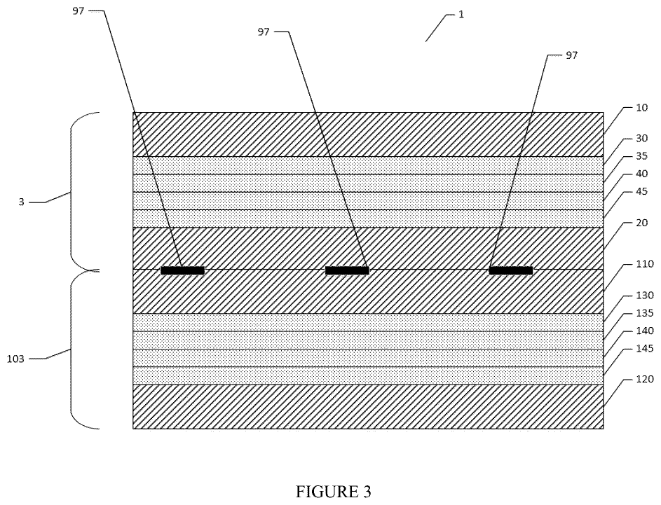

FIG. 3 illustrates one particular backsheet 1 according to the general depiction of FIG. 2. As shown in FIG. 3, the first nonwoven fabric 3 may include outer spunbond layers 10,20 with four (4) interior fine-fiber containing layers (e.g., meltblown layer) 30,35,40,45. The backsheet 1 also includes the second nonwoven fabric 103 that includes two outer spunbond layers 110,120 with four (4) interior fine-fiber containing layers (e.g., meltblown layer) 130,135,140,145. Thermal bonds 97 attach the first nonwoven fabric 1 and the second nonwoven fabric 103 together. As noted above, although the thermal bond sites 97 are illustrated as shallow bond sites, the thermal bond sites may extend through the entire thickness of the backsheet or completely through one of the nonwoven fabrics and only partially through the other nonwoven fabric.

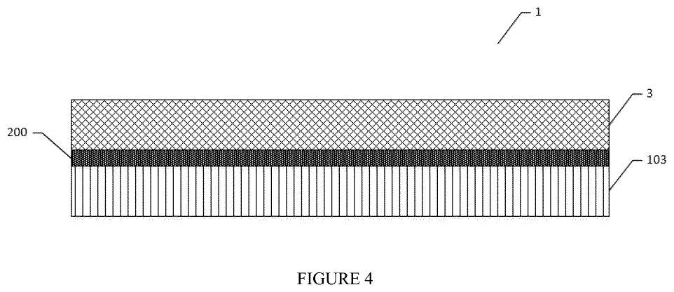

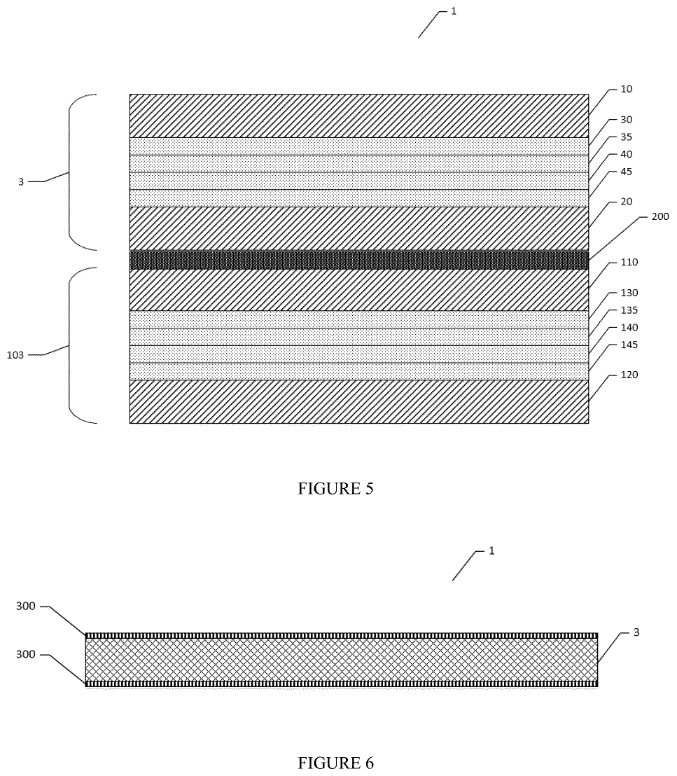

FIG. 4 illustrates a backsheet 1 including a first nonwoven fabric 1 and a second nonwoven fabric 103 bonded together via an adhesive layer 200. FIG. 5 illustrates one particular backsheet 1 according to the general depiction of FIG. 4. FIG. 5 illustrates the first nonwoven fabric 3 including outer spunbond layers 10,20 with four (4) interior fine-fiber containing layers (e.g., meltblown layer) 30,35,40,45. The backsheet 1 also includes the second nonwoven fabric 103 that includes two outer spunbond layers 110,120 with four (4) interior fine-fiber containing layers (e.g., meltblown layer) 130,135,140,145. Adhesive layer 200 bonds the first nonwoven fabric 3 and the second nonwoven fabric 103 together.

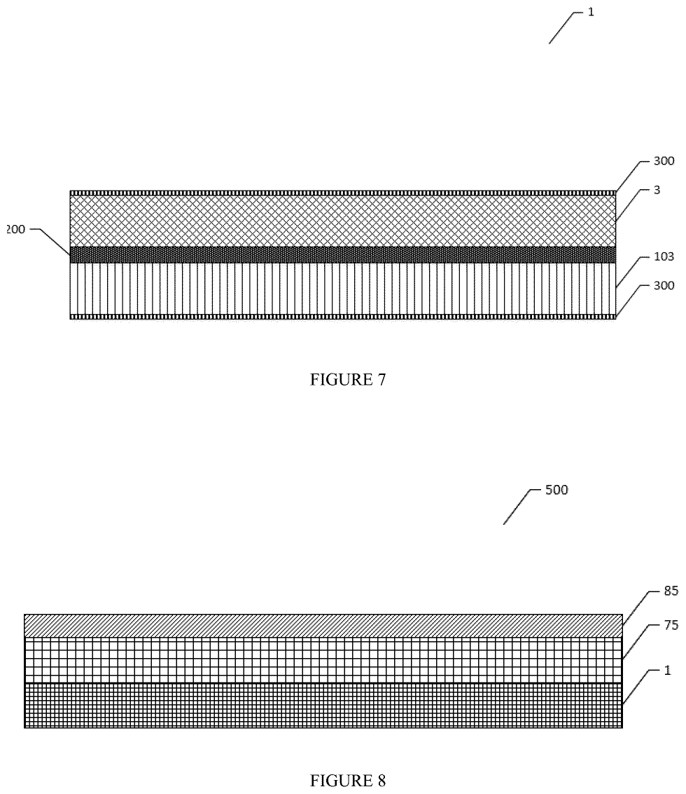

FIG. 6 illustrates a backsheet 1 formed from the first nonwoven fabric 3 and a water-repellant coating 300 applied to both sides thereof in accordance with certain embodiments of the invention. FIG. 7 illustrates a backsheet 1 formed from the first nonwoven fabric 3 and the second nonwoven fabric 103 bonded together by adhesive layer 200. The backsheet 1 includes a water-repellant coating 300 applied to both sides thereof in accordance with certain embodiments of the invention.

In another aspect, the present invention provides a hygiene article including (i) a topsheet, (ii) an absorbent core, and (iii) a backsheet, such as any of those disclosed and described herein, in which the absorbent core is located directly or indirectly between the topsheet and the backsheet. FIG. 8, for instance, illustrates a cross-sectional view of a hygiene article 500 including a backsheet 1, such as any of those described and disclosed herein, an absorbent core 75, and a topsheet 86, wherein the absorbent core is located between the backsheet and the topsheet. By way of example only, the hygiene article may be a diaper, a pull-up under-pant, an under-pad, or a sanitary napkin.

In another aspect, the present invention provides A method of making a backsheet comprising forming at least a first nonwoven fabric, in which forming the first nonwoven fabric may comprise the following steps: (i) providing or forming a first outermost spunbond layer including a first plurality of continuous spunbond fibers; (ii) providing or forming a second outermost spunbond layer including a second plurality of continuous spunbond fibers; (iii) providing or forming a first fine-fiber containing layer including a first plurality of fine fibers; (iv) providing or forming a second fine-fiber containing layer including a second plurality of fine fibers; (v) positioning the first fine-fiber containing layer proximately or adjacent to the first outermost spunbond layer, and positioning the second fine-fiber containing layer is proximately or adjacent to the second outermost spunbond layer and adjacent to the first fine-fiber containing layer to form a first intermediate material; and (vi) consolidating the first intermediate material to form a backsheet, such as those disclosed and described herein.

In accordance with certain embodiments of the invention, the step of consolidating the first intermediate material may comprise subjecting the first intermediate material to a thermal bonding operation or ultrasonic bonding operation and imparting one or more bond sites. The method may also comprise a step of providing or forming a second nonwoven fabric, such as those described and disclosed herein, and bonding the second nonwoven fabric to the first nonwoven fabric, such as by any consolidation method disclosed herein. Additionally or alternatively, the method may comprise a step of applying a water-repellent coating onto a first outermost surface, a second outermost surface, or both of the backsheet.

### EXAMPLES

The present disclosure is further illustrated by then following examples, which in no way should be construed as being limiting. That is, the specific features described in the following examples are merely illustrative and not limiting.

A prototype modeling was built to simulate a baby wearing a diaper (pants) that falls on its butt and sits on the floor for a period of time. There is impact force while sitting down and constant pressure while sitting on the ground. Assuming a baby of 11 kg weight, while sitting down, we can use the theory of free fall to calculate impact force.

### Impact Force Calculation:

| | Baby | Unit | Data |
|---|---|---|---|
| Values | Weight - m | kg | 11 |
| | Sitting down(½ height) - h | meter | 0.5 |
| | Time to touch ground - t | Sec | 0.3 |
| | Touch area - S | cm² | 177 |
| Results | Impact force - F₁ | N | 115.95 |
| | Pressure - P₁ | mbar | 65.51 |

### Constant Pressure Calculation:

| | Baby | Unit | Data |
|---|---|---|---|
| Values | Weight - m | kg | 8.8 |
| | Touch area - S | cm² | 177 |
| Results | Constant force - F₂ | N | 88 |
| | Constant pressure - P₂ | mbar | 49.72 |

The designed backsheet should have liquid barrier higher than 65.51 mbar, and keep 30 min for constant test. To validate there is no liquid leakage during these activities, liquid penetration of backsheet is evaluated through hydrohead (e.g., hydrostatic head) testing. The backsheet is challenged at pressure of 70 mbar with the rate of increasing of water pressure set as 500 mbar/min and then challenged at pressure of 50 mbar for 30 min. The testing agent is saline water (0.9% NaCl water solution). The foregoing is referred to as Impact Test #1.

Besides liquid barrier, softness is another important feature of disposable products for babycare and femcare applications. Handle-O-Meter is a common softness tester for sheeted material such as textile, nonwoven, tissue, etc. Eventually the softness is characterized as Handle-O-Meter(HOM). A lower HOM value indicates better softness. In this design, HOM may be set below 18 grams in Machine Direction (MD) and 10 grams in Cross Direction (CD) based on acceptance criteria by multiple hygiene customers.

### Example 1—SMS Fabric with Surface Treatment

The backsheet consisted of a single nonwoven fabric having a basis weight of 45 gsm with a SMMMMS construction that was subjected to surface treatment.

### Raw Materials:

Spunbond Layers: Polypropylene resin 3155E5 with a MFR of 35 g/10 min;
Meltblown Layers: Polypropylene resin HP461Y with a MFR of 1400 g/10 min;
Softness Enhancer in the Spunbond Layers: Vistamaxx 6202 (propylene-based elastomer including isotactic propylene repeat units and random ethylene distribution with 15% by weight of ethylene content), 5% by weight;
Slip Agent: A016, 0.5% by weight;
White master batch: WF802, 0.5% by weight;
Process: Spinning, Calender bonding, Finishing, drying;
Lab testing results: See Tables 1A and 1B

TABLE 1A
The SMMMMS with surface treatment, the
properties (IST80.6, 0.9% NaCl water)

| | Raising speed-500 mbar/ | | | | 500 mbar/min to 70 |
|---|---|---|---|---|---|
| | min, 1st drop water/mbar | | | | mbar; Constant pressure |
| Item | 1 | 2 | 3 | AVG | 50 mbar - 30 min |
| Hydro Head | 87.2 | 81.1 | 75.2 | 81.17 | No leakage |

TABLE 1B
Other Properties

| Item | Unit | Test method | Results |
|---|---|---|---|
| Air permeability | cfm | IST70.1 | 20.99 |
| Pore size | μm\| | PMI | 6~8 |
| Alcohol repellent | Rating | IST80.8 | 5 |
| Mason Jar | min | IST80.5 | >120 |

### Example 2—SMS Fabric without Surface Treatment

The backsheet consisted of a two nonwoven fabrics adhesively bonded together, in which the first nonwoven fabric had a basis weight of 25 gsm with a SMMMMS construction and the second nonwoven fabric has a basis weight of 35 gsm with a SMMMMS construction. The backsheet was devoid of any surface treatment.

### Raw Materials:

Spunbond Layers: Polypropylene resin HH350F with a MFR of 35 g/10 min;
Meltblown Layers: Polypropylene resin HP461Y with a MFR of 1400 g/10 min;
Softness Enhancer in the Spunbond Layers: Vistamaxx 6202 (propylene-based elastomer including isotactic propylene repeat units and random ethylene distribution with 15% by weight of ethylene content), 12%, increase the ratio to improve the softness
Slip Agent: A016, 2%
White master batch: WF802, 0.5%
Process: Spinning, Calender bonding
Lab testing results: See Tables 2A-2B

TABLE 2A
SMMMMS without surface treatment, the properties
(IST80.6, 0.9% NaCl water, 25 gsm + 35 gsm)

| | | | | | Water pressure - |
|---|---|---|---|---|---|
| Item | | | | | 70 mbar |
| 25 gsm | Rate of increase of water pressure- | | | | (500 mbar/min) |
| laminated | 500 mbar/min, 1st water drop/mbar | | | | Constant pressure |
| with 35 gsm | 1 | 2 | 3 | AVG | 50 mbar - 30 min |
| Hydro Head | 108 | 114 | 102 | 108 | No leakage |

TABLE 2B
Other Properties

| | | | | | Laminated |
|---|---|---|---|---|---|
| | | Test | | | 25 gsm + |
| Item | Unit | method | 25 gsm | 35 gsm | 35 gsm |
| Air permability | cfm | IST 70.1 | 56.8 | 35.0 | 23.0 |
| Pore size diameter | μm | PMI | 8~12 | 7~11 | 5~10 |
| HOM-MD | g | IST 90.3 | 8.1 | 16.7 | 27.3 |
| HOM-CD | g | IST 90.3 | 4.9 | 8.0 | 16.3 |

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A backsheet, comprising: at least a first nonwoven fabric comprising—
(i) a first outermost spunbond layer including a first plurality of continuous spunbond fibers;
(ii) a second outermost spunbond layer including a second plurality of continuous spunbond fibers;
(iii) a first fine-fiber containing layer including a first plurality of fine fibers; wherein the first fine-fiber containing layer is located proximately or adjacent to the first outermost spunbond layer; and
(iv) a second fine-fiber containing layer including a second plurality of fine fibers; wherein the second fine-fiber containing layer is located proximately or adjacent to the second outermost spunbond layer and adjacent to the first fine-fiber containing layer;
wherein a total amount of fine fibers comprising the first plurality of fine fibers and the second plurality of fine fibers account for about 15 to about 40% by weight of the first nonwoven fabric.

2. The backsheet of claim 1, wherein a total amount of continuous spunbond fibers comprising the first plurality of continuous spunbond fibers and the second plurality of continuous spunbond fibers account for about 60 to about 85% by weight of the first nonwoven fabric.

3. The backsheet of claim 1, wherein first outermost spunbond layer, the second outermost spunbond layer, or both independently from each other comprises a spunbond polymeric composition including (i) a spunbond-polymer component, and (ii) a spunbond-additive component.

4. The backsheet of claim 3, wherein the spunbond-polymer component comprises from 60 to 100% by weight of one or more non-elastomeric polymers.

5. The backsheet of claim 3, wherein the spunbond-additive component comprises a softness enhancer comprising an elastomeric polymer, wherein the first plurality of continuous spunbond fibers, the second plurality of continuous spunbond fibers, or both independently from each other have from about 2 to about 20% by weight of the elastomeric polymer.

6. The backsheet of claim 3, wherein the first nonwoven fabric has from about 0.01 to about 5% by weight of the elastomeric polymer.

7. The backsheet of claim 3, wherein the spunbond-additive component further comprises a slip additive comprising an amide, wherein (i) the first plurality of continuous spunbond fibers, the second plurality of continuous spunbond fibers, or both independently from each other have from 0.1 to 5% by weight of the amide, and/or (ii) the first nonwoven fabric has from about 0.005 to about 0.5% by weight of the amide.

8. The backsheet of claim 1, wherein the first plurality of fine fibers, the second plurality of fine fibers, or both independently from each other comprise meltblown fibers, melt-fibrillated fibers, or electrospun fibers.

9. The backsheet of claim 8, wherein the first fine-fiber containing layer, the second fine-fiber containing layer, or both independently from each other comprises a fine-fiber polymeric composition including (i) a fine-fiber-polymer component comprising one or more non-elastomeric polymers, and (ii) a fine-fiber-additive component comprising a softness enhancer comprising one or more elastomeric polymers.

10. The backsheet of claim 9, wherein the fine-fiber-polymer component comprises from 60 to 100% by weight of the one or more non-elastomeric polymers, and the fine-fiber-additive component comprises from about 0 to about 20% by weight of the one or more elastomeric polymers.

11. The backsheet of claim 1, wherein the first nonwoven fabric has a basis weight from about 10 to about 100 gsm.

12. The backsheet of claim 1, further comprising a second nonwoven fabric bonded to the first nonwoven fabric by one or more thermal bond sites and/or via a layer of adhesive including an adhesive composition located between the first nonwoven fabric and the second nonwoven fabric.

13. The backsheet of claim 12, wherein the adhesive layer has a discontinuous pattern having one or more regions devoid of the adhesive composition.

14. The backsheet of claim 1, further comprising (i) a water-repellent surface treatment deposited onto a first outermost surface, a second outermost surface, or both, and/or (ii) the presence of masterbatch comprising a masterbatch additive including a water-repellent compound(s).

15. A hygiene article, comprising:
(i) a topsheet;
(ii) an absorbent core; and
(iii) a backsheet according to claim 1; wherein the absorbent core is located directly or indirectly between the topsheet and the backsheet.

16. The hygiene article of claim 15, wherein the hygiene article is a diaper, a pull-up under-pant, an under-pad, or a sanitary napkin, and wherein the backsheet is devoid of a film.

17. A method of making a backsheet, comprising:
(i) forming at least a first nonwoven fabric comprising—
(a) providing or forming a first outermost spunbond layer including a first plurality of continuous spunbond fibers;
(b) providing or forming a second outermost spunbond layer including a second plurality of continuous spunbond fibers;
(c) providing or forming a first fine-fiber containing layer including a first plurality of fine fibers;
(d) providing or forming a second fine-fiber containing layer including a second plurality of fine fibers;
(e) positioning the first fine-fiber containing layer proximately or adjacent to the first outermost spunbond layer, and positioning the second fine-fiber containing layer is proximately or adjacent to the second outermost spunbond layer and adjacent to the first fine-fiber containing layer to form a first intermediate material; and
(f) consolidating the first intermediate material to form a backsheet.

18. The method of claim 17, wherein consolidating the first intermediate material comprises subjecting the first intermediate material to a thermal bonding operation or ultrasonic bonding operation.

19. The method of claim 17, further comprising a step of providing or forming a second nonwoven fabric, and bonding the second nonwoven fabric to the first nonwoven fabric.

20. The method of claim 17, further comprising a step of (i) applying a water-repellent coating onto a first outermost surface, a second outermost surface, or both of the backsheet, and/or (ii) adding a masterbatch comprising a masterbatch additive including a water-repellent compound(s) into a polymer melt forming the fibers of the backsheet.
